(19) United States
(12) Patent Application Publication
(10) Pub. No.: US 2015/0283317 A1
(43) Pub. Date: Oct. 8, 2015

(54) APPARATUS FOR NITRIC OXIDE DELIVERY TO A PATIENT AND METHODS OF USING SAME

(71) Applicant: BAYLOR COLLEGE OF MEDICINE, Houston, TX (US)

(72) Inventors: Paul A. Checchia, Houston, TX (US); Ronald A. Bronicki, Houston, TX (US)

(21) Appl. No.: 14/439,449

(22) PCT Filed: Oct. 25, 2013
(86) PCT No.: PCT/US13/66954

§ 371 Date: Apr. 29, 2015

Related U.S. Application Data
(60) Provisional application No. 61/719,795, filed on Oct. 29, 2012.

(51) Int. Cl. A61M 1/36

(52) U.S. Cl. CPC A61M 1/3603; A61M 1/3666; A61M 2202/0208; A61M 2202/0275

(57) ABSTRACT

Nitric oxide delivery devices comprise a chamber (33) carrying a gas transfer member (40). An interior chamber (44) of the gas transfer member (40) is in fluid communication with a gas source (60, 62) and the outer wall surface of the gas transfer member (40) is in fluid communication with blood flowing through the chamber (33). The gas transfer member (40) permits the gas to pass through or diffuse through the member from the interior chamber (44) to the chamber (33) carrying the blood so that the blood becomes infused with the gas. Nitric oxide and oxygen can be diffused through the same gas transfer member. Alternatively, nitric oxide can first be passed through a first gas transfer member and into the blood followed by oxygen being passed through a second gas transfer member and into the blood. Alternatively, oxygen can first be infused into the blood followed by nitric oxide.

### BACKGROUND

1. Field of Invention

The invention is directed to medical devices and systems for delivering nitric oxide to a patient's blood, and in particular, medical devices for delivery of nitric oxide to a patient's blood as it circulates through an extracorporeal circulation system such that may be established during surgery.

2. Description of Art

Cardiac surgery requiring cardiopulmonary bypass (“CPB”) generally requires establishment of a circulation system outside of the body to facilitate circulation of blood through the patient, as well as oxygenation of the blood. Such systems are known in the art and are generally referred to as cardiopulmonary bypass machines that are operated by trained technicians referred to as perfusionists. In addition to monitoring the oxygen levels in the blood, the perfusionist can also monitor other blood chemistry and blood temperature and modify both as desired or necessary to assist in the surgery. Modification of the blood chemistry can be accomplished by devices, such as an oxygenator, that deliver oxygen to the blood.

### SUMMARY OF INVENTION

Broadly, the medical devices or systems and methods disclosed herein are directed to nitric oxide delivery devices having a chamber carrying a gas transfer member. An interior of the gas transfer member is in fluid communication with a gas source and the outer wall surface of the gas transfer member is in fluid communication with blood flowing through a chamber. The gas transfer member permits the gas to pass through or diffuse through the gas transfer member from the interior chamber to the chamber carrying the blood so that the blood becomes infused with the gas.

In one specific embodiment, one gas transfer member is in fluid communication with both a nitric oxide source and an oxygen source so that both nitric oxide and oxygen are diffused into the chamber carrying the blood.

In other specific embodiments, the nitric oxide delivery device includes two gas transfer members. In one such embodiment having two gas transfer members, nitric oxide is the first gas to be diffused into the blood through a first gas transfer member and oxygen is the second gas to be diffused into the blood through a second gas transfer member. In an alternative embodiment, oxygen is the first gas to be diffused into the blood through a first gas transfer member and nitric oxide is the second gas to be diffused into the blood through a second gas transfer member.

It is believed that the present devices will effectively deliver nitric oxide to a patient's blood during cardiopulmonary bypass surgery to result in a significantly shortened duration of mechanical ventilation [8.4+7.6 hours vs. 16.3+6.5 hours (p<0.05)] and intensive care unit length of stay [53.8+19.7 hours vs. 79.4+37.7 hours (p<0.05)] as compared to a patient who does not receiving nitric oxide during surgery. In addition, it is believed that delivery of nitric oxide to a patient's blood during cardiopulmonary bypass surgery also can lower troponin levels at 12, 24, and 48 hours (p<0.05), lower B-type natriuretic peptide levels at 12 and 24 hours (p<0.05), and lower the use of diuretics. Further, it is believed that delivery of nitric oxide to a patient's blood during cardiopulmonary bypass surgery also can result in the patient having a higher mean hemoglobin at 48 hours despite no differences in chest tube output, PRBC transfusion, platelet counts or transfusion, FFP transfusion, or pT/pTT in the first 48 hours after surgery. Accordingly, it is believed that delivery of nitric oxide to a patient's blood during cardiopulmonary bypass surgery will result in myocardial protection, improved fluid balance, and improved postoperative ICU course. It is to be understood, however, that the effects and results of the nitric oxide delivery devices disclosed herein are dependent upon the skill and training of the operators and surgeons.

### BRIEF DESCRIPTION OF DRAWINGS

FIG. 1 is a partial cross-sectional/partial schematic view of one specific embodiment of a nitric oxide delivery device disclosed herein.

FIG. 2 is a partial cross-sectional/partial schematic view of another specific embodiment of a nitric oxide delivery device disclosed herein.

FIG. 3 is a partial cross-sectional/partial schematic view of an additional specific embodiment of a nitric oxide delivery device disclosed herein.

While the invention will be described in connection with the preferred embodiments, it will be understood that it is not intended to limit the invention to that embodiment. On the contrary, it is intended to cover all alternatives, modifications, and equivalents, as may be included within the spirit and scope of the invention as defined by the appended claims.

### DETAILED DESCRIPTION OF INVENTION

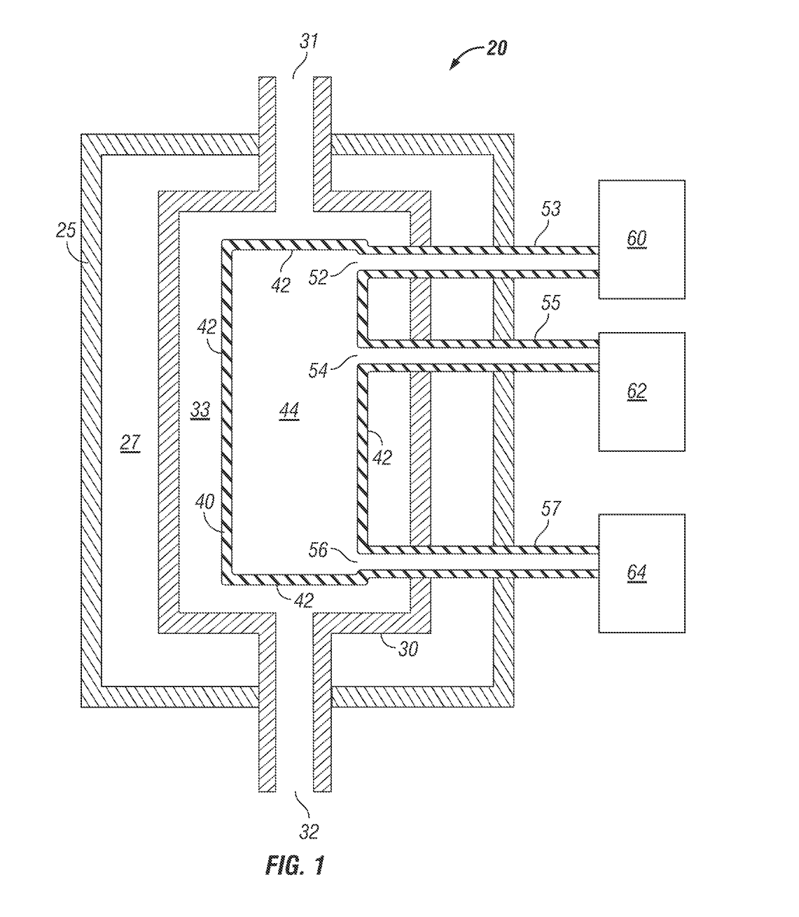

Referring now to Figures, nitric oxide delivery devices are designed to deliver nitric oxide to blood flowing through an extracorporeal circulation system. In one embodiment illustrated in FIG. 1, nitric oxide delivery device 20 comprises housing 25 defining chamber 27 having disposed therein blood flow housing 30. Blood flow housing 30 include inlet 31, outlet 32 and chamber 33. Disposed within chamber 33 is gas transfer member 40 having one or more walls 42 defining interior chamber 44. As shown in FIG. 1, gas transfer member 40 is a rectangular-shaped member having four walls 42. It is to be understood, however, that gas transfer member 40 can be spherical-shaped or any other shape having as few as one wall such as in the case of a sphere, or any other number of walls depending on the shape of gas transfer member 40.

Gas transfer member 40 can be any device capable of allowing gases such as nitric oxide, oxygen and the like to pass through wall(s) 42 of gas transfer member 40, but prevent blood (not shown) from flowing through wall(s) 42 of gas transfer member 40. In the embodiment of FIG. 1, gas transfer member 40 comprises two inlets 52, 54, and one outlet 56.

Inlet 52 is in fluid communication with interior chamber 44 and tubing 53 which is in fluid communication with nitric oxide source 60. Thus, inlet 52 delivers nitric oxide to interior chamber 44 of gas transfer member 40 so that it can then diffuse through wall(s) 42 of gas transfer member 40 and combine with blood (not shown) flowing through chamber 33 of blood flow housing 30. Nitric oxide source 60 can be any type of nitric oxide source known in the art and can include additional components, such as a regulator, a monitor, and/or a titration component, to facilitate delivery of the desired amount of nitric oxide to interior chamber 44.

Inlet 54 is in fluid communication with interior chamber 44 and tubing 55 which is in fluid communication with oxygen source 62. Thus, inlet 54 delivers oxygen to interior chamber 44 of gas transfer member 40 so that it can then diffuse through wall(s) 42 of gas transfer member 40 and combine with blood (not shown) flowing through chamber 33 of blood flow housing 30. Oxygen source 62 can be any type of oxygen source known in the art and can include additional components, such as a regulator, a monitor, and/or a titration component, to facilitate delivery of the desired amount of oxygen to interior chamber 44.

Outlet 56 is in fluid communication with interior chamber 44 and tubing 57 which is in fluid communication with venting device 64 to facilitate removal of excess nitric oxide and/or oxygen from interior chamber 44. Venting device 64 can be any type of gas collection system.

Although the embodiment of FIG. 1 shows a single outlet 56, it is to be understood that more than one outlet 56 can be included as desired or necessary to remove excess oxygen or nitric oxide from interior chamber 44 of gas transfer member 40. Similarly, one or more additional inlets can be included as desired or necessary to deliver oxygen and/or nitric oxide to interior chamber 44 of gas transfer member 40. Moreover, a single inlet can deliver both oxygen and nitric oxide to interior chamber 44 in the embodiment of FIG. 1.

In operation of the embodiment of FIG. 1, an extracorporeal circulation system is established by having blood from a patient flow from the body, through the system, and back into the patient's body. As noted above, such systems are known in the art and generally involve use of a pump or other device operated by a perfusionist. As the blood flows through the extracorporeal circulation system, the blood flows into inlet 31, into chamber 33, and out of outlet 32. As the blood flows through chamber 33 it is infused with nitric oxide and oxygen flowing through walls 42 of gas transfer member 40 as a result of both gases flowing from their respective sources 60, 62. As a result, in this embodiment, the blood from the patient is infused with both nitric oxide and oxygen within chamber 33. Excess nitric oxide and oxygen flow out of outlet 56, through tubing 57, and into venting device 64.

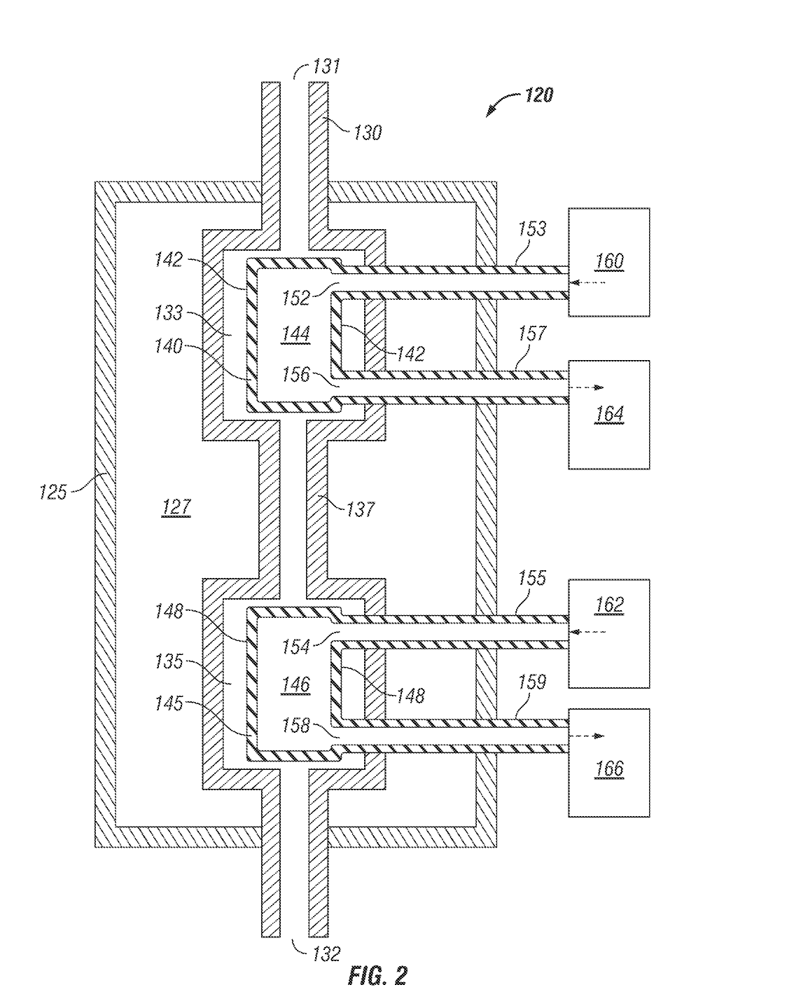

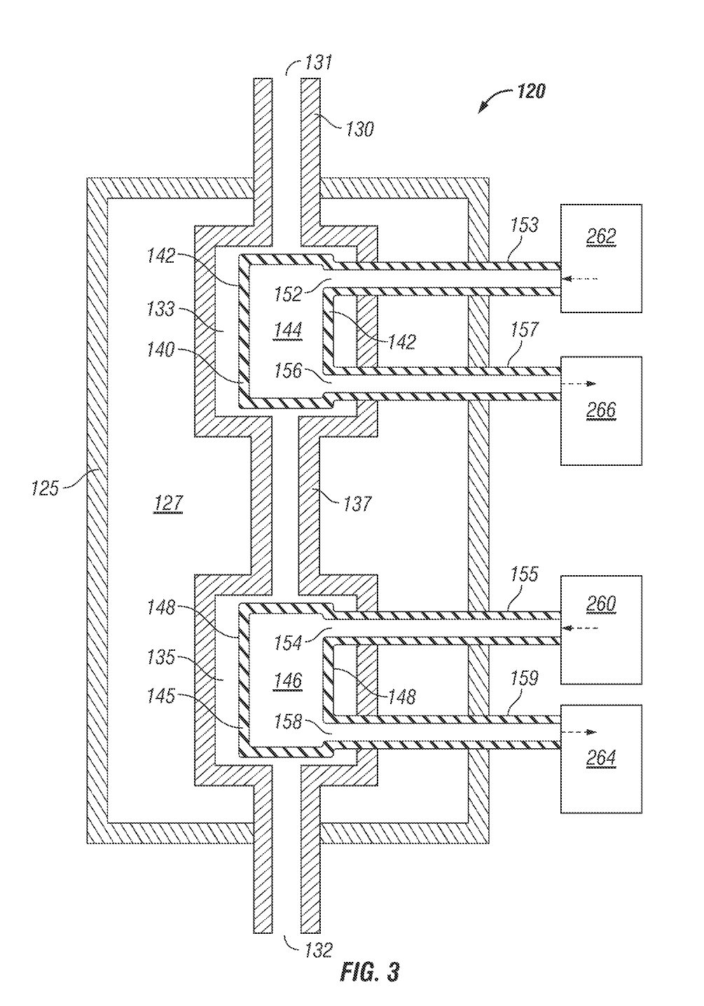

Referring now to FIGS. 2-3, in two other specific embodiments, nitric oxide delivery device 120 comprises housing 125 defining chamber 127 having disposed therein blood flow housing 130. Blood flow housing 130 include inlet 131, outlet 132 and first chamber 133, second chamber 135, and passage 137 placing first chamber 133 in fluid communication with second chamber 135. Disposed within chamber 133 is first gas transfer member 140 having one or more walls 142, and disposed within chamber 135 is second gas transfer member 145 having one or more walls 148.

As shown in FIG. 2, gas transfer members 140, 145 are rectangular-shaped members having four walls 142, 148, respectively. It is to be understood, however, that gas transfer members 140, 145 can be spherical-shaped or any other shape having as few as one wall such as in the case of a sphere, or any other number of walls depending on the shape of gas transfer members 140, 145. In addition, gas transfer members 140, 145 can be any device capable of allowing gases such as nitric oxide, oxygen and the like to pass through wall(s) 142, 148, but prevent blood (not shown) from flowing through wall(s) 142, 148.

With respect to the embodiment of FIG. 2, gas transfer member 140 comprises inlet 152 and outlet 156. Inlet 152 is in fluid communication with interior chamber 144 and tubing 153 which is in fluid communication with nitric oxide source 160. Thus, inlet 152 delivers nitric oxide to interior chamber 144 of gas transfer member 140 so that it can then diffuse through wall(s) 142 of gas transfer member 140 and combine with blood (not shown) flowing through chamber 133 of blood flow housing 130. Nitric oxide source 160 can be any type of nitric oxide source known in the art and can include additional components, such as a regulator, a monitor, and/or a titration component, to facilitate delivery of the desired amount of nitric oxide to interior chamber 144.

Outlet 156 is in fluid communication with interior chamber 144 and tubing 157 which is in fluid communication with venting device 164. Venting device 164 can be any type of gas collection system.

Inlet 154 is in fluid communication with interior chamber 146 and tubing 155 which is in fluid communication with oxygen source 162. Thus, inlet 154 delivers oxygen to interior chamber 146 of gas transfer member 145 so that it can then diffuse through wall(s) 148 of gas transfer member 145 and combine with blood (not shown) flowing through chamber 135 of blood flow housing 130. Oxygen source 160 can be any type of oxygen source known in the art and can include additional components, such as a regulator, a monitor, and/or a titration component, to facilitate delivery of the desired amount of oxygen to interior chamber 146.

Outlet 158 is in fluid communication with interior chamber 146 and tubing 159 which is in fluid communication with venting device 166 to facilitate removal of excess oxygen from interior chamber 146. Venting device 166 can be any type of gas collection system.

In operation of the embodiment of FIG. 2, an extracorporeal circulation system is established by having blood from a patient flow from the body, through the system, and back into the patient's body. As the blood flows through the extracorporeal circulation system, the blood flows into inlet 131, into chamber 133, through passage 137, into chamber 135, and out of outlet 132. As the blood flows through chamber 133 it is infused with nitric oxide flowing through walls 142 of gas transfer member 140 as a result of nitric oxide flowing from nitric oxide source 160. Excess nitric oxide flows out of outlet 156, through tubing 157, and into venting device 164. Therefore, in this embodiment, the blood from the patient is first infused with nitric oxide within chamber 133.

After being infused with nitric oxide in chamber 133, the blood then flows through passage 137 and into chamber 135. As the blood flows through chamber 135 it is infused with oxygen flowing through walls 148 of gas transfer member 145 as a result of oxygen flowing from oxygen source 162. Excess oxygen flows out of outlet 158, through tubing 159 and into venting device 166. Therefore, in this embodiment, the blood from the patient is infused with oxygen within chamber 135 after being infused with nitric oxide within chamber 133. The blood then flows out of outlet 135 so that can be carried back to the patient.

Referring now to the embodiment of FIG. 3 which is substantially similar to the embodiment of FIG. 2 and, therefore, includes like reference numerals, gas transfer member 140 comprises interior chamber 144 in fluid communication with inlet 152, tubing 153, outlet 156, and tubing 157 similar to the embodiment of FIG. 2. In the embodiment of FIG. 3, however, oxygen source 262 is in fluid communication with tubing 153, inlet 152, and, thus, interior chamber 144. Accordingly, inlet 152 delivers oxygen to interior chamber 144 of gas transfer member 140 so that it can then diffuse through wall(s) 142 of gas transfer member 140 and combine with blood (not shown) flowing through chamber 133 of blood flow housing 130. Oxygen source 262 can be any type of oxygen source known in the art and can include additional components, such as a regulator, a monitor, and/or a titration component, to facilitate delivery of the desired amount of oxygen to interior chamber 144.

Outlet 156 is in fluid communication with venting device 266 by tubing 157 to facilitate removal of excess oxygen from interior chamber 144. Venting device 266 can be any type of gas collection system.

Similar to the embodiment of FIG. 2, inlet 154 and tubing 155 are in fluid communication interior chamber 146 of gas transfer member 145; however, instead of being in fluid communication with an oxygen source as shown in FIG. 2, inlet 154 and tubing 155 and, therefore, interior chamber 146, are in fluid communication with nitric oxide source 260. Accordingly, inlet 154 delivers nitric oxide to interior chamber 146 of gas transfer member 145 so that it can then diffuse through wall(s) 148 of gas transfer member 145 and combine with blood (not shown) flowing through chamber 135 of blood flow housing 130. Nitric oxide source 260 can be any type of nitric oxide source known in the art and can include additional components, such as a regulator, a monitor, and/or a titration component, to facilitate delivery of the desired amount of nitric oxide to interior chamber 146.

Outlet 158 is in fluid communication with venting device 264 by tubing 159 to facilitate removal of excess nitric oxide from interior chamber 146. Venting device 264 can be any type of gas collection system.

In operation of the embodiment of FIG. 3, the blood is infused with oxygen prior to being infused with nitric oxide. Thus, in the embodiment of FIG. 3, as the blood flows through chamber 133 it is infused with oxygen flowing through walls 142 of gas transfer member 140 as a result of oxygen flowing from oxygen source 262. Excess oxygen flows out of outlet 156, through tubing 157, and into venting device 266. Therefore, in this embodiment, the blood from the patient is first infused with oxygen within chamber 133.

After being infused with oxygen in chamber 133, the blood then flows through passage 137 and into chamber 135. As the blood flows through chamber 135 it is infused with nitric oxide flowing through walls 148 of gas transfer member 145 as a result of nitric oxide flowing from nitric oxide source 260. Excess nitric oxide flows out of outlet 158, through tubing 159, and into venting device 264. Therefore, in this embodiment, the blood from the patient is infused with nitric oxide within chamber 135 after being infused with oxygen within chamber 133. The blood then flows out of outlet 135 so that can be carried back to the patient.

Infusion of nitric oxide to a patient's blood during cardiopulmonary bypass surgery has been found by the inventors to result in a significantly shortened duration of mechanical ventilation [8.4+7.6 hours vs. 16.3+6.5 hours (p<0.05)] and intensive care unit length of stay [53.8+19.7 hours vs. 79.4+37.7 hours (p<0.05)] as compared to a patient not receiving nitric oxide during surgery. The inventors have also observed that delivery of nitric oxide to a patient's blood during cardiopulmonary bypass surgery can lower troponin levels at 12, 24, and 48 hours (p<0.05), lower B-type natriuretic peptide levels at 12 and 24 hours (p<0.05), and lower the use of diuretics. In addition, the inventors have found that delivery of nitric oxide to a patient's blood during cardiopulmonary bypass surgery also can result in the patient having a higher mean hemoglobin at 48 hours despite no differences in chest tube output, PRBC transfusion, platelet counts or transfusion, FFP transfusion, or pT/pTT in the first 48 hours after surgery. Accordingly, the inventors believe that delivery of nitric oxide to a patient's blood during cardiopulmonary bypass surgery will result in myocardial protection, improved fluid balance, and improved postoperative ICU course.

It is to be understood that the invention is not limited to the exact details of construction, operation, exact materials, or embodiments shown and described, as modifications and equivalents will be apparent to one skilled in the art. For example, inlets 52 and 54 can be combined into a single inlet. Moreover, the shapes and sizes of the housing, chambers, and gas transfer members can be any shape or size desired or necessary to facilitate the infusion of nitric oxide and oxygen to the blood flowing through the devices. In addition, in the embodiments of FIGS. 2 and 3, passage 137 is not required, but instead chambers 133, 135 can be separated by a wall instead of passage 137. Alternatively, the gas transfer members 140, 145 can be disposed in series in the same chamber. Moreover, although the embodiments of FIGS. 2-3 are shown as having first and second chambers 133, 135, and passage 137, it is to be understood that all of first chamber 133, second chamber 135, and passage 137 can comprise a single chamber having two separate portions separated by a passage. Additionally, the devices of FIGS. 1-3 are not required to include housing 25, 125. Further, luer locks or other connectors can be included to facilitate connection of inlets 31, 131 and outlets 32, 132 of housings 30, 130 to additional components making up the extracorporeal circulation system. Similarly, luer locks or other connections can be used to facilitate connection of the sources of nitric oxide and oxygen to the nitric oxide delivery devices. Accordingly, the invention is therefore to be limited only by the scope of the appended claims.

What is claimed is:

1. A nitric oxide delivery device for infusing blood with nitric oxide, the device comprising:
a housing having a chamber; and
a gas transfer member disposed within the chamber, the gas transfer member being in fluid communication with a nitric oxide source and an oxygen source,
wherein nitric oxide and oxygen enter the gas transfer member simultaneously.

2. A nitric oxide delivery device for infusing blood with nitric oxide, the device comprising:
a housing having a chamber;
a first gas transfer member disposed within the chamber, the first gas transfer member being in fluid communication with a first gas source; and
a second gas transfer member disposed within the chamber in series with the first gas transfer member, the second gas transfer member being in fluid communication with a second gas source,
wherein the first gas source comprises a first gas, and the second gas source comprises a second gas, the second gas being different from the first gas, and
wherein blood flowing through the chamber contacts the first gas transfer member before contacting the second gas transfer member.

3. The nitric oxide delivery device of claim 2, wherein the first gas is nitric oxide and the second gas is oxygen.

4. The nitric oxide delivery device of claim 2, wherein the first gas is oxygen and the second gas is nitric oxide.

5. The nitric oxide delivery device of claim 2, wherein the chamber comprises a first portion and a second portion, the first portion in fluid communication with the second portion through a passageway, the first gas transfer member being disposed in the first portion and the second gas transfer member being disposed in the second portion.

6. The nitric oxide delivery device of claim 5, wherein the first gas is nitric oxide and the second gas is oxygen.

7. The nitric oxide delivery device of claim 5, wherein the first gas is oxygen and the second gas is nitric oxide.

8. A method of delivering nitric oxide to a blood stream, the method comprising the steps of:
(a) flowing blood through a first chamber of a housing;
(b) flowing nitric oxide from a nitric oxide source into a first gas transfer member disposed within the first chamber of the housing causing the nitric oxide to pass through the first gas transfer member and into the blood; and
(c) flowing oxygen from an oxygen source causing the oxygen to be infused into the blood.

9. The method of claim 8, wherein during step (c), the oxygen flows from the oxygen source into the first gas transfer member disposed within the chamber causing the oxygen to pass through the gas transfer member and into the blood.

10. The method of claim 8, wherein oxygen flows from the oxygen source through a second gas transfer member disposed within a second chamber of the housing causing the oxygen to pass through the second gas transfer member and into the blood.

11. The method of claim 10, wherein step (b) occurs prior to step (c).

12. The method of claim 10, wherein step (c) occurs prior to step (b).
